(19) United States
(12) Patent Application Publication
(10) Pub. No.: US 2003/0139761 A1
(43) Pub. Date: Jul. 24, 2003

(54) Catheter system for use in stent implantation

(76) Inventors: Ib Jorgensen, Haigerloch (DE); Randolf Von Oepen, Tubingen (DE)

Correspondence Address: SHINJYU GLOBAL IP COUNSELORS, LLP, 1233 20TH STREET, NW, SUITE 700, WASHINGTON, DC, 20036-2680, US

(21) Appl. No.: 10/296,252

(22) PCT Filed: May 22, 2001
(86) PCT No.: PCT/EP01/05893

(30) Foreign Application Priority Data
May 22, 2000 (DE) 200092049

(51) Int. Cl. A61M 29/00

(52) U.S. Cl. 606/194

(57) ABSTRACT

The present invention relates to a catheter arrangement comprising a guide wire tube 3 for receiving a guide wire 2 and an expandable balloon 7 fastened to the guide wire tube 3, the distal end 5 of the guide wire tube 3 projecting beyond the distal end of the balloon 7, the distal end 5 of the guide wire tube 3 being designed as a soft tip.

### BACKGROUND OF THE INVENTION

1. Field of the Invention

The present invention relates to a catheter arrangement for stent implantation. More specifically, the present invention relates to a catheter arrangement having a guide wire tube for receiving a guide wire, and an expandable balloon fastened to the guide wire tube, with the distal end of the guide wire tube projecting beyond the distal end of the balloon.

2. Background Information

Nowadays, catheter arrangements which are provided at their distal end with a balloon that can be expanded are used at narrowed places in body vessels or body cavities for expanding the narrowed portion. A stent which can be placed in the narrowed portion for stabilizing the vessel wall can additionally be crimped onto said expandable balloon. The catheter arrangement is then guided with the help of a guide wire to the narrowed place in the patient's body, and the narrowed portion is expanded by expansion of the balloon and the crimped stent is placed in the narrowed portion.

### SUMMARY OF THE INVENTION

It is therefore the object of the present invention to provide a catheter arrangement of the type indicated in the preamble of claim 1 which shows a very high flexibility at the distal end portion.

This object is achieved by a catheter arrangement comprising: a guide wire tube for receiving a guide wire, and an expandable balloon fastened to the guide wire tube. The distal end of the guide wire tube projects beyond the distal end of the balloon. The distal end of the guide wire tube is designed as a soft tip.

In accordance with one aspect of the present invention, the catheter arrangement has a first part of the guide wire tube made from a first material and a second part of the guide wire tube from a second material. Preferably, the second material has a higher flexibility than the first material. The distal end of the guide wire tube is made from the second material having a higher flexibility.

In accordance with another aspect of the present invention, a transition portion of the guide wire tube which forms the transition from the first part to the second part of the guide wire tube is arranged at the distal side of the balloon.

In accordance with another aspect of the present invention, the transition portion of the guide wire tube is arranged at the proximal side of the balloon.

In accordance with another aspect of the present invention, the transition portion of the guide wire tube is arranged in the inner cavity of the balloon.

In accordance with another aspect of the present invention, the length of the distal end of the guide wire tube which projects from the balloon n the direction of the guide wire is greater than a length of a distal fastening portion between the balloon and the guide wire tube.

The quality of a balloon catheter is distinguished in that the catheter can follow vessel sections wound in a way that is as simple as possible. Therefore, when the distal point, generally called “tip”, is preferably made from a more flexible material specifically suited therefor, the whole system will preferably follow the windings of the vessel. At the same time, however, it must be ensured that the catheter can reliably be pushed forwards into the narrowed portions of the vessel. This property is described by the technical term “pushability”. However, if the whole catheter system is made from soft materials, this has a contra-indicative effect.

Preferably, the guide wire tube is made from two composed parts, the two parts consisting of different materials. The part of the guide wire tube that forms the distal end is here made in a particularly advantageous way from a material having a higher elasticity or flexibility than the other part. This results in a particularly flexible design of the distal end of the guide wire tube or the catheter arrangement. The catheter arrangement is thereby made more flexible while maintaining its “pushability” at the same time.

Methods, such as gluing or welding (laser, contact and hot-air welding as well as inductive welding) are inter alia suitable for connecting the two parts of the guide wire tube.

According to a preferred embodiment of the present invention a transition portion of the guide wire tube from the part made from a first material to the part made from a second material is arranged at the distal side of the balloon. The distal fastening position of the balloon is thus directly arranged above the transition portion of the guide wire tube. According to another preferred embodiment of the present invention, said transition portion of the guide wire tube is arranged in the area of the balloon or according to a third preferred embodiment at the proximal side of the balloon. This results in a particularly high elasticity of the catheter arrangement because the part of the guide wire tube made from the very flexible material has a relatively great length.

Further details, features and advantages of the invention will become apparent from the following description of embodiments taken in conjunction.

### BRIEF DESCRIPTION OF THE DRAWINGS

Referring now to the attached drawings which form a part of this original disclosure:

FIG. 1 is a schematically strongly simplified sectional view of a catheter arrangement of the invention according to a first embodiment of the present invention;

FIG. 2 is a schematically strongly simplified sectional view of a catheter arrangement of the invention according to a second embodiment of the present invention; and

FIG. 3 is a schematically strongly simplified sectional view of a catheter arrangement of the invention according to a third embodiment of the present invention.

### DETAILED DESCRIPTION OF THE PREFERRED EMBODIMENTS

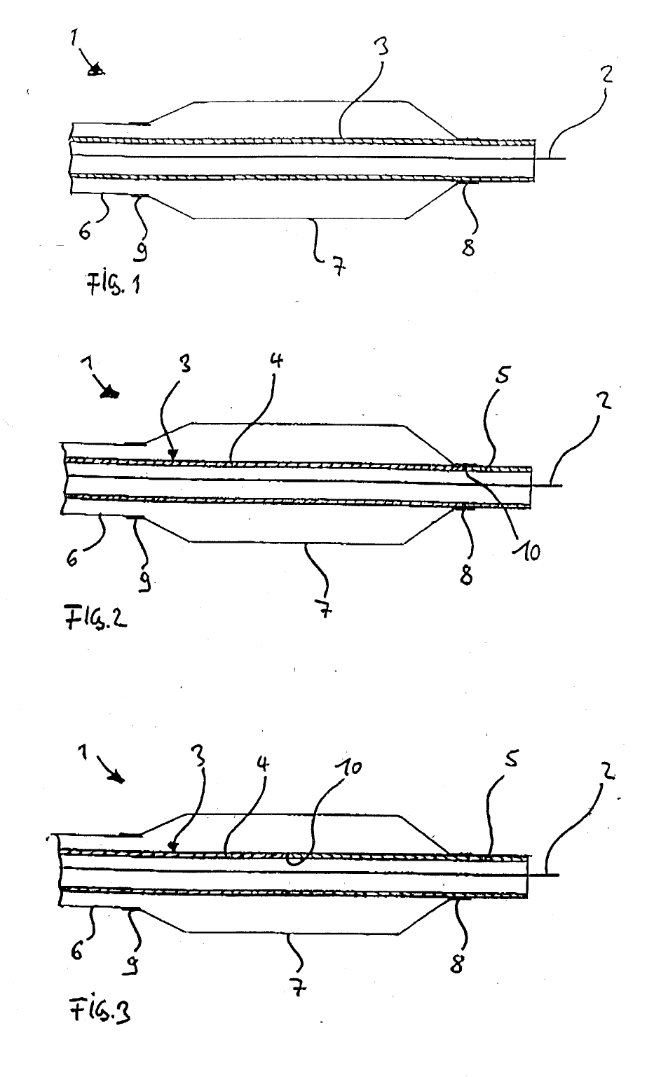

Referring initially to FIG. 1, a catheter arrangement 1 for implantation of a stent is illustrated in accordance with a first embodiment of the present invention. The catheter arrangement 1 comprises a guide wire tube 3 having a guide wire 2 arranged therein. The guide wire 2 and the guide wire tube 3 together form a guide means.

As shown in FIG. 1, the catheter arrangement 1 further includes a balloon 7 and a tube 6. The balloon 7 is fastened with its distal end via a distal fastening portion 8 to the guide wire tube 3 and with its proximal end via a proximal fastening portion 9 to the tube 6 (cf. FIG. 1). In this context “distal end” means “leading towards the heart” and “proximal end” “leading away from the heart”. The tube 6 which is connected to the balloon 7 serves to guide a suitable medium, such as a saline solution, into the hollow interior of the balloon 7 and to expand the same at the narrowed place. In this way a stent (not shown) provided around the balloon 7 can be expanded, and the stent, in turn, is able to stabilize the expansion of the narrowed place in the hollow body vessel as a vessel support.

As can be seen from FIG. 1, the distal fastening portion 8 of the balloon 7 is arranged on the guide wire tube 3 slightly offset in a proximal direction from the distal end of the guide wire tube. As a result, the connection place between the balloon 7 and the guide wire tube 4 is offset from the end of the guide wire tube 3.

In contrast to known catheter arrangements, the catheter arrangement according to the invention has a soft tip portion whose flexibility or elasticity is greater than that of the remaining portion of the guide wire tube 4. This helps to prevent injuries to the patient during insertion of the catheter arrangement.

FIG. 2 shows a second embodiment according to the present invention. Like parts are provided with the same reference numerals as in the first embodiment.

In contrast to the first embodiment, the second embodiment shown in FIG. 2 includes a guide wire tube 3 which consists of two parts 4 and 5. The two parts 4 and 5 are here made from different materials. The part 5 of the guide wire tube 3 which forms the distal end of the guide wire tube 3 is here made from a more elastic material than the part 4 of the guide wire tube 3. Hence, the distal end of the guide wire tube 3 has a high flexibility or elasticity. This is even enhanced by the fact that the distal fastening portion 8, to which the balloon 7 is connected with the guide wire tube 3, is offset from the end of the guide wire tube 3.

As shown in FIG. 2, a transition portion 10 is arranged between the two parts 4 and 5 of the guide wire tube 3 directly on the distal fastening portion 8 or at the distal side of the fastening portion 8.

If the transition portion 10 is directly arranged on the distal fastening portion 8, the connection of the two parts 4 and 5 on the transition portion 10 is additionally secured by the distal fastening portion 8 between the balloon 7 and the guide wire tube 3.

FIG. 3 shows a third embodiment of a catheter arrangement according to the invention. Like parts are again designated with the same reference numerals as in the two preceding embodiments.

In contrast to the second embodiment, the transition portion 10 from the distal part 5 of the guide wire tube 3 to the proximal part 4 of the guide wire tube 3 is arranged in the third embodiment in the interior of the balloon 7 or at the proximal side of the balloon 7. Since the distal part 5 of the guide wire tube 3 is made from a more flexible material than the proximal part 4, a relatively long section of the guide wire tube 3 is made from a very flexible material. This results in a very high flexibility of the catheter arrangement 1.

1. Catheter arrangement comprising
a guide wire tube (3) for receiving a guide wire (2), and
an expandable balloon (7) fastened to said guide wire tube (3), the distal end (5) of said guide wire tube (3) projecting beyond the distal end of said balloon (7),
characterized in
that said distal end (5) of said guide wire tube (3) is designed as a soft tip.

2. The catheter arrangement according to claim 1, characterized in that a part (4) of said guide wire tube (3) is made from a first material and a part (5) of said guide wire tube (3) from a second material.

3. The catheter arrangement according to claim 2, characterized in that said second material has a higher flexibility than said first material.

4. The catheter arrangement according to claim 3, characterized in that said distal end (5) of said guide wire tube (3) is made from said second material having a higher flexibility.

5. The catheter arrangement according to any one of claims 2 to 4, characterized in that a transition portion (10) of said guide wire tube (3) which forms the transition from said first part (4) to said second part (5) of said guide wire tube (3) is arranged at the distal side of said balloon (7).

6. The catheter arrangement according to any one of claims 2 to 4, characterized in that said transition portion (10) of said guide wire tube (3) is arranged at the proximal side of said balloon (7).

7. The catheter arrangement according to any one of claims 2 to 4, characterized in that said transition portion (10) of said guide wire tube (3) is arranged in the inner cavity of said balloon (7).

8. The catheter arrangement according to any one of claims 1 to 7, characterized in that the length of said distal end (5) of said guide wire tube (3) which projects from said balloon (7) in the direction of said guide wire (2) is greater than a length of a distal fastening portion (8) between said balloon (7) and said guide wire tube (3).
